(19) United States
(12) Patent Application Publication
(10) Pub. No.: US 2026/0015354 A1
(43) Pub. Date: Jan. 15, 2026

(54) Solid State Forms of Ensifentrine

(71) Applicant: Cipla Limited, Mumbai (IN)

(72) Inventors: Arijit Das, Mumbai (IN); Ramanaiah Chennuru, Velegala Ponnuru, Andhra Pradesh (IN); Ramesh Devarapalli, Chatrai, Andhra Pradesh (IN); Anjaneyaraju Indukuri, Hyderabad (IN); Botcha Yerramnaidu, Battupadu, Andhra Pradesh (IN); Shrikant Suresh Mudgal, Panvel, Maharashtra (IN); Manohar Raghunath Surve, Karjat, Maharashtra (IN); Maruti Laxman Ghaitadkar, New Panvel, Maharashtra (IN)

(73) Assignee: Cipla Limited, Mumbai (IN)

(21) Appl. No.: 19/139,777

(22) PCT Filed: Jun. 21, 2023
(86) PCT No.: PCT/IN2023/050591

§ 371 Date: Jun. 16, 2025

(30) Foreign Application Priority Data
Dec. 14, 2022 (IN) 202221072216

(51) Int. Cl. C07D 471/04; A61K 31/4985

(52) U.S. Cl. CPC C07D 471/04; A61K 31/4985

(57) ABSTRACT

The present invention relates to solid state forms of Ensifentrine and methods for preparation. use and isolation of such compounds and novel process for the preparation of Ensifentrine. The present invention further relates to an improved. cost effective and industrially viable process for preparation of Ensifentrine.

### FIELD OF THE INVENTION

The present invention is related to novel solid state forms of Ensifentrine, the process of preparation of such novel solid state forms, pharmaceutical compositions, and use thereof. The present invention further relates to an improved, cost effective and industrially viable process for preparation of Ensifentrine.

### BACKGROUND OF THE INVENTION

Ensifentrine is a first-in-class, inhaled, dual inhibitor of the phosphodiesterase 3 (PDE3) and phosphodiesterase 4 (PDE4) enzymes. This dual inhibition enables it to combine bronchodilator and anti-inflammatory properties in one compound, differentiating it from existing drug classes used to treat COPD, including corticosteroids, beta2-agonists and anti-muscarinics.

Ensifentrine also activates the Cystic Fibrosis Transmembrane Conductance Regulator (CFTR), which is beneficial in reducing mucous viscosity and improving mucociliary clearance. This potentially makes it an attractive therapy for the treatment of cystic fibrosis. Ensifentrine can also be used for the treatment of asthma, COVID-19 and other respiratory diseases.

Ensifentrine is described in U.S. Pat. Nos. 6,794,391; 7,378,424; and 7,105,663 (incorporated by reference herein in its entirety) and other publications, as a compound RPL-554 or (N-{2-[(2E)-2-(mesitylimino)-9,10-dimethoxy-4-oxo-6,7-dihydro-2H-pyrimido[6,1-a]-isoquinolin-3 (4H)-yl]ethyl}urea).

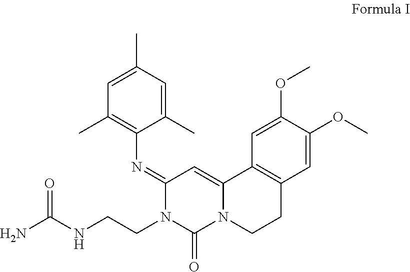 [SMILES: COc1cc2c(cc1OC)-c1c/c(=N\c3c(C)cc(C)cc3C)n(CCNC(N)=O)c(=O)n1CC2]

Prior art suggests that Ensifentrine exists in the polymorphic forms.

U.S. Pat. No. 9,062,047 B2; (incorporated by reference herein in its entirety) discloses Form I, Form II, Form III, Form IV and Form V.

The pharmaceutical industry is often confronted with the phenomenon of multiple polymorphs of the same crystalline chemical entity. The discovery of new polymorphic forms, new salts, solid state forms, and solvates of a pharmaceutically useful compound provides a new opportunity to improve the performance characteristics of a pharmaceutical product such as stability, solubility, bioavailability, etc. It enlarges the repertoire of materials that a formulation scientist has available for designing, for example, a pharmaceutical dosage form of a drug with a targeted release profile, a different crystal habit, higher crystallinity, or polymorphic stability, or solubility, which may offer better processing or handling characteristics, improved dissolution profile, or improved shelf-life.

Therefore, there is a continuous need in the art to develop stable and soluble solid state forms of Ensifentrine, which are having greater stability, flowability, dissolution properties, compressibility, density, increased potency or, lack of toxicity thereby increasing the bioavailability as well as to enhance the efficacy, of the parent molecule in lower doses.

Further, the present inventors observed that the process for preparation of free Ensifentrine base as disclosed in U.S. Pat. No. 6,794,391 is not industrially and commercially viable. There remains a need therefore to provide an improved process for synthesis of Ensifentrine which is cost effective and industrially viable.

### SUMMARY OF THE INVENTION

Accordingly, the invention is directed to novel solid state forms of Ensifentrine, methods of preparing such solid state forms, and methods of treating Chronic obstructive pulmonary disease (COPD) with such novel solid state forms. The novel solid state forms may be a crystalline, amorphous or a co-crystal.

In a first aspect, the present invention provides novel crystalline forms of Ensifentrine, hereinafter referred to as “Form C1”, “Form C2” and “Form C3”.

The polymorphic forms of Ensifentrine, may be in a pseudo polymorphic form. Accordingly, pseudo polymorphs are provided that includes hydrates and/or solvates.

In an embodiment, the crystalline nature of forms according to the present invention is characterized by X-ray powder diffraction.

In a second aspect, the present invention provides processes for preparing novel solid state forms of Ensifentrine thereof.

In a third aspect, the present invention provides a pharmaceutical composition comprising novel solid state forms of Ensifentrine and processes for the preparation of the novel composition.

In a fourth aspect, the present invention provides use of the novel solid state forms Ensifentrine for the preparation of a pharmaceutical composition comprising an effective amount of the novel solid state forms Ensifentrine of the present invention and at least one pharmaceutically acceptable excipient. Such pharmaceutical composition may be administered to a mammalian patient in any dosage form, e.g. tablet, capsule, suspension, syrup, DPI, MDI, nebulizer, nasal spray etc.

In a fifth aspect, the present invention is directed to methods of treating and/or preventing respiratory disorders such as cystic fibrosis, asthma and chronic obstructive pulmonary disease (COPD) by administering a therapeutically effective amount of a novel solid state forms of Ensifentrine thereof.

In a sixth aspect, the present invention is directed to the use of novel solid state forms of Ensifentrine in the manufacture of a medicament for treating and/or preventing respiratory disorders such as cystic fibrosis, asthma and chronic obstructive pulmonary disease (COPD).

In a seventh aspect, the present invention provides an improved process for the preparation of free Ensifentrine base, wherein said process comprises the steps of:
a) Condensing 2-Chloro-9,10-dimethoxy-6,7-dihydro-4H-pyrimido[6,1-a]isoquinolin-4-one with 2,4,6-trimethylaniline in a suitable solvent at a temperature in the range of 75 to 90° C. to obtain (E)-2-(mesitylimino)-9,10-dimethoxy2,3,6,7-tetrahydro-4H-pyrimido[6,1-a]isoquinolin-4-one;
b) Condensing (E)-2-(mesitylimino)-9,10-dimethoxy2,3,6,7-tetrahydro-4H-pyrimido[6,1-a]isoquinolin-4-one with N-(2-bromoethyl) phthalimide of step a) in a suitable solvent and in presence of catalyst and a base at a temperature in the range of 105 to 125° C. to (E)-2-(2-(2-(mesitylimino)-9,10-dimethoxy-4-oxo-6,7-dihydro-2H-pyrimido[6,1-a] isoquinolin-3(4H)-yl) ethyl) isoindoline-1,3-dione;
c) Purifying the material by dissolving the material obtained in step b) in water and alcohol mixture, followed by addition of base, stirring at a temperature in the range of 20 to 35° C. to obtain the purified (E)-2-(2-(2-(mesitylimino)-9,10-dimethoxy-4-oxo-6,7-dihydro-2H-pyrimido[6,1-a] isoquinolin-3 (4H)-yl) ethyl) isoindoline-1,3-dione;
d) Reacting purified (E)-2-(2-(2-(mesitylimino)-9,10-dimethoxy-4-oxo-6,7-dihydro-2H-pyrimido [6,1-a] isoquinolin-3(4H)-yl) ethyl) isoindoline-1,3-dione of step c) with 40% methyl amine in suitable solvent at a temperature in the range of 35 to 50° C. to obtain (E)-3-(2-aminoethyl)-2-(mesitylimino)-9,10-dimethoxy-2,3,6,7-tetrahydro-4H-pyrimido[6,1-a] isoquinolin-4-one; and
e) Reacting (E)-3-(2-aminoethyl)-2-(mesitylimino)-9,10-dimethoxy-2,3,6,7-tetrahydro-4H-pyrimido[6,1-a]isoquinolin-4-one of step d) with sodium cyanate in suitable solvent and acid at a temperature in the range of 60 to 75° C. to obtain Ensifentrine.

In an eight aspect, free Ensifentine is converted to its crystalline “C1”, “C2” and “C3” polymorphic forms of Ensifentine.

### BRIEF DESCRIPTION OF THE DRAWINGS

FIG. 1 is the X-ray powder diffraction pattern (XRPD) of the crystalline Form C1 of Ensifentrine.

FIG. 2 is the Differential scanning calorimetry (DSC) of the crystalline Form C1 of Ensifentrine.

FIG. 3 is the Thermogravimetry analysis (TGA) of the crystalline Form C1 of Ensifentrine.

FIG. 4 is the Infrared Spectrum (IR) of the crystalline Form C1 of Ensifentrine.

FIG. 5 is comparative solubility of Form C1 and Form I at pH 3.

FIG. 6 is the X-ray powder diffraction pattern (XRPD) of the crystalline Form C2 of Ensifentrine obtained as per example 2.

FIG. 7 is the X-ray powder diffraction pattern (XRPD) of the crystalline Form C2 of Ensifentrine obtained as per example 3.

FIG. 8 is the Differential scanning calorimetry (DSC) of the crystalline Form C2 of Ensifentrine.

FIG. 9 is the Thermogravimetry analysis (TGA) of the crystalline Form C2 of Ensifentrine.

FIG. 10 is the X-ray powder diffraction pattern (XRPD) of the crystalline Form C3 of Ensifentrine.

FIG. 11 is the Differential scanning calorimetry (DSC) of the crystalline Form C3 of Ensifentrine.

FIG. 12 is the Thermogravimetry analysis (TGA) of the crystalline Form C3 of Ensifentrine.

FIG. 13 is the Infrared Spectrum (IR) of the crystalline Form C3 of Ensifentrine.

FIG. 14 is comparative solubility of Form C3 and Form I at pH 3.

### DETAILED DESCRIPTION

The present invention relates to novel solid state forms of Ensifentrine.

An embodiment of the invention is directed to crystalline forms of Ensifentrine having one or more of the characteristics as defined below.

The crystalline forms of Ensifentrine could be in a hydrated or solvated or anhydrous form.

As used herein, the term “hydrate” is understood as a substance that is formed by adding water molecules. The skilled person will appreciate that the water molecules are absorbed, adsorbed or contained within a crystal lattice of the solid compounds, usually in defined stoichiometric ratio. The notation for a hydrated compound may be nH₂O, where n is the number of water molecules per formula unit of the compound. For example, in a hemihydrate, n is 0.5; in a monohydrate n is one; in a sesquihydrate, n is 1.5; in a dihydrate, n is 2; and so on.

As used herein, the term “solvated” is understood to mean formation of a complex of variable stoichiometry comprising Ensifentrine of Formula (I) and a solvent. Such solvents for the purpose of the invention may not interfere with the biological activity of the solute. Typically, the solvent used is a pharmaceutically acceptable solvent. Examples of suitable pharmaceutically acceptable solvents include but not limited to C1-C4 alcohol solvents, Methylene dichloride (MDC), acetone, methyl benzoate, ethyl acetate, benzyl alcohol, 4-methyl pentane 2-one, propylene glycol, acetonitrile, 1,3-dioxane, and dimethylsulfoxide (DMSO), and solvates of more than 1%.

As used herein interchangeably, Ensifentrine free base or Ensifentrine is understood comprising Ensifentrine of Formula (I) prepared by method known in the art, such as the one described in U.S. Pat. No. 6,794,391 which is incorporated herein by reference or using the novel process of the present invention.

The solvate can be isolated either as an amorphous form or in a crystalline form, preferably in a crystalline form.

The solvate can be further isolated either in anhydrous form or hydrated form.

In one embodiment of the invention, the present invention provides a crystalline “Form C1” of Ensifentrine which has good flow characteristics.

The crystalline Form C1 is a relatively stable towards moisture and humidity, thereby representing a crystalline form of Ensifentrine, thus enhancing the efficacy of the parent molecule in lower doses.

The crystalline Form C1 according to the present invention may be characterized by powder X-ray diffraction.

The Crystalline Form C1 may be characterized by having an XRPD pattern comprising at least two peaks selected from 4.5, 7.7, 8.9, 12.8, 14.5, 19.3, 22.4 and 23.5±0.2° 2θ.

The X-ray powder diffractogram may be as depicted in FIG. 1.

Optionally, the crystalline Form C1 of Ensifentrine may be further characterized by data selected from the group comprising of: an X-ray powder diffraction pattern having at least two peaks selected from 4.5, 7.7, 8.9, 12.8, 14.5,19.3, 22.4 and 23.5±0.2° 2θ; an XRPD pattern as depicted in FIG. 1; and combinations thereof.

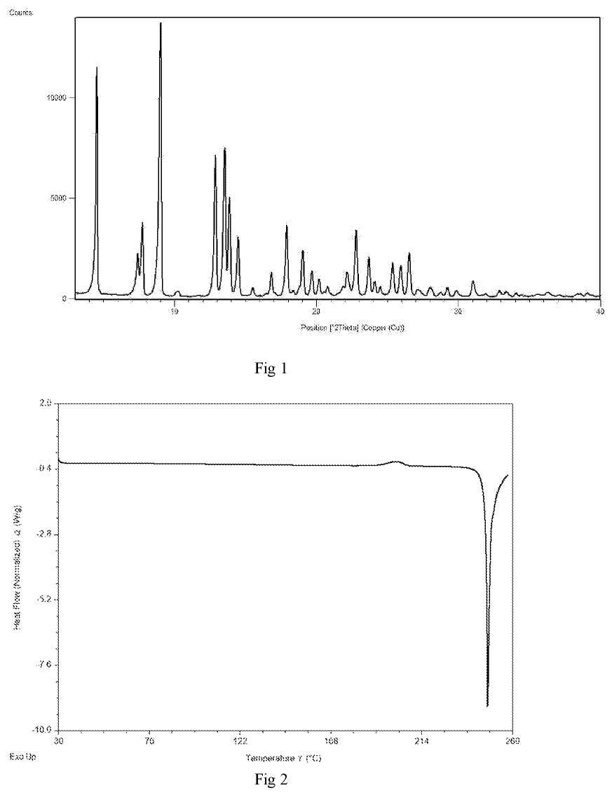

The crystalline Form C1 of Ensifentrine may be further characterized by Differential scanning calorimetry (DSC) depicted in FIG. 2.

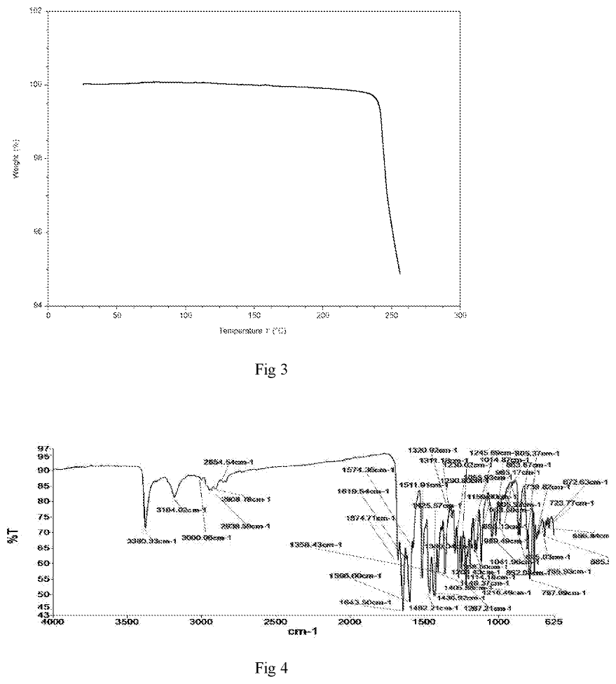

The crystalline Form C1 of Ensifentrine may be further characterized by Thermogravimetry analysis (TGA) is as depicted in FIG. 3.

The crystalline Form C1 of Ensifentrine may be further characterized by Infrared spectrometry is as depicted in FIG. 4.

The solubility of Form C1 and Form I disclosed in U.S. Pat. No. 9,062,047 at pH 3 were compared. The result obtained is provided below in Table 1. The data clearly indicate that the novel Form C1 has better solubility compared to Form I. The graph of solubility is shown in FIG. 5.

TABLE 1

| | |
|---|---|
| Time | % Drug released (mg/mL) |

| (min) | Form I | Form C3 |
|---|---|---|
| 15 | 1.38 | 2.11 |
| 30 | 1.39 | 1.96 |
| 60 | 1.31 | 2.00 |
| 120 | 1.35 | 2.05 |
| 240 | 1.39 | 2.13 |

In another embodiment, the present invention provides a crystalline “Form C2” of Ensifentrine which has good flow characteristics.

The crystalline Form C2, according to the present invention may be characterized by powder X-ray diffraction.

The Crystalline Form C2 may be characterized by having an XRPD pattern comprising at least two peaks selected from 5.2, 9.3, 10.6, 13.9 and 22.2±0.2° 2θ.

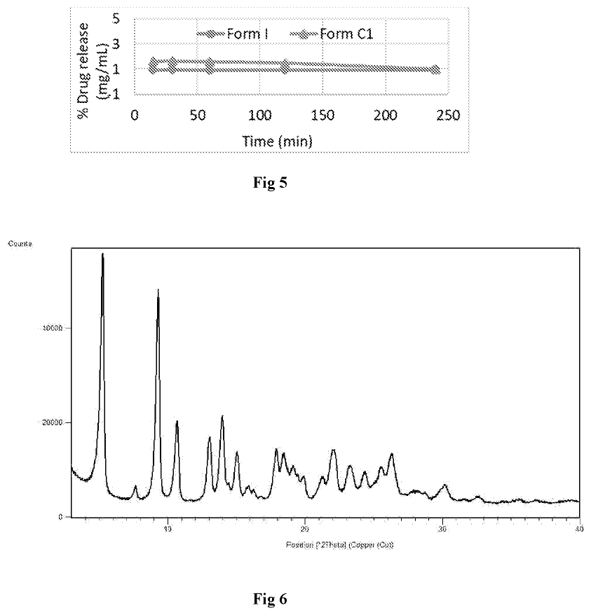

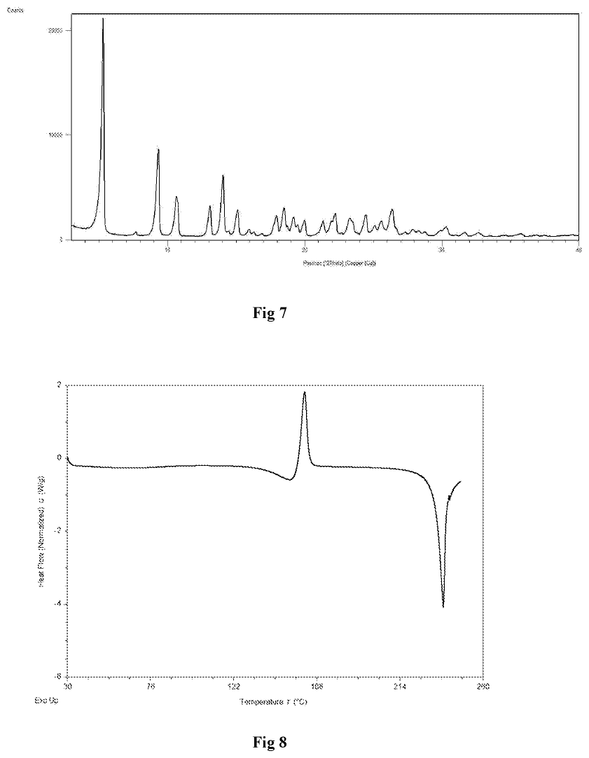

The X-ray powder diffractogram may be as depicted in FIGS. 6 and 7.

Optionally, the crystalline Form C2 of Ensifentrine may be further characterized by data selected from the group comprising of: an X-ray powder diffraction pattern having at least two peaks selected from 5.2, 9.3, 10.6, 13.9 and 22.2+0.2° 2θ; an XRPD pattern as depicted in FIG. 6; an XRPD pattern as depicted in FIG. 7; and combinations thereof.

The crystalline Form C2 of Ensifentrine may be further characterized by Differential scanning calorimetry (DSC) depicted in FIG. 8.

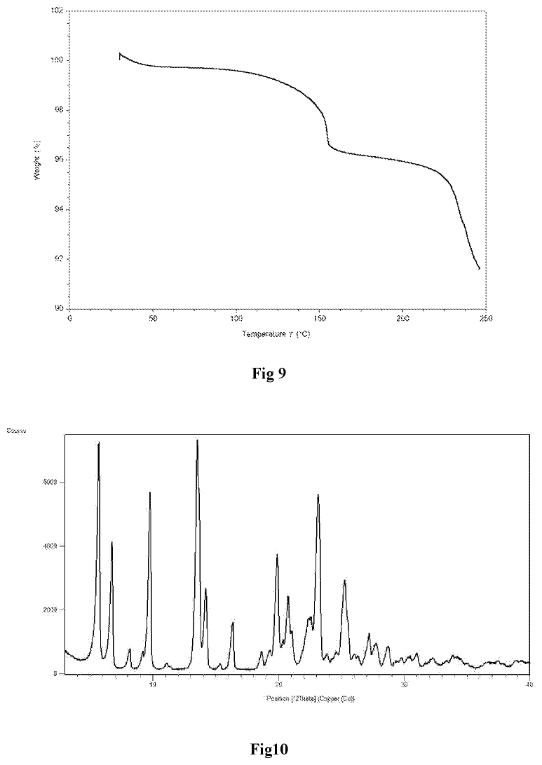

The crystalline Form C2 of Ensifentrine may be further characterized by Thermogravimetry analysis (TGA) is as depicted in FIG. 9.

In another embodiment of the invention, the present invention provides a crystalline “Form C3” of Ensifentrine which has good flow characteristics.

The crystalline Form C3 is relatively stable towards moisture and humidity, thereby representing a crystalline form of Ensifentrine, thus enhancing the efficacy of the parent molecule in lower doses.

The crystalline Form C3 according to the present invention may be characterized by powder X-ray diffraction.

The X-ray powder diffractogram may be as depicted in FIG. 10.

Optionally, the crystalline Form C3 of Ensifentrine may be further characterized by data selected from the group comprising of: an X-ray powder diffraction pattern having at least two peaks selected from 5.6, 6.6, 9.7, 13.5 and 16.3±0.2° 2θ; an XRPD pattern as depicted in FIG. 10; and combinations thereof.

The crystalline Form C3 of Ensifentrine may be further characterized by Differential scanning calorimetry (DSC) depicted in FIG. 11.

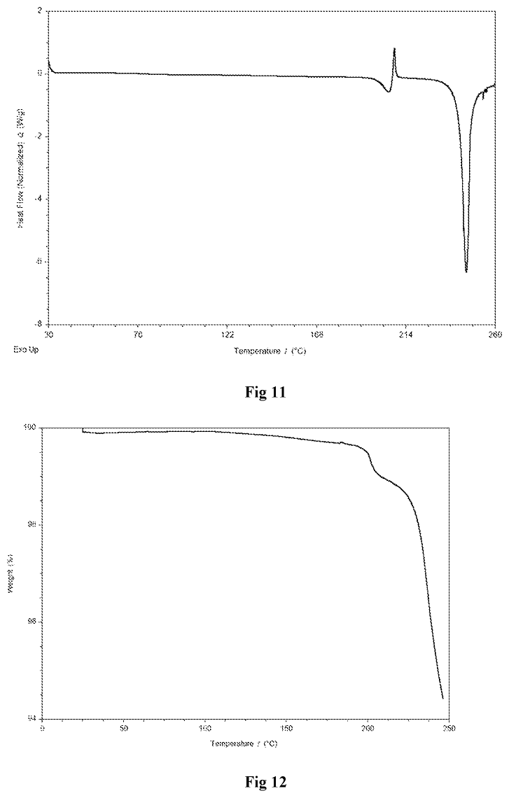

The crystalline Form C3 of Ensifentrine may be further characterized by Thermogravimetry analysis (TGA) is as depicted in FIG. 12.

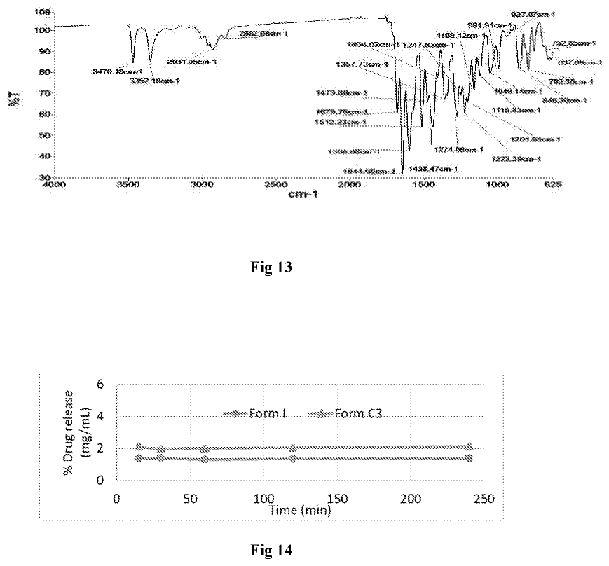

The crystalline Form C3 of Ensifentrine may be further characterized by Infrared spectrometry as depicted in FIG. 13.

The solubility of Form C3 and Form I disclosed in U.S. Pat. No. 9,062,047 at pH 3 was compared. The result obtained is provided below in Table 2. The data clearly indicates that the novel Form C3 has better solubility compared to Form I. The graph of solubility is shown in FIG. 14.

TABLE 2

| | |
|---|---|
| Time | % Drug released (mg/mL) |

| (min) | Form I | Form C3 |
|---|---|---|
| 15 | 1.38 | 2.11 |
| 30 | 1.39 | 1.96 |
| 60 | 1.31 | 2.00 |
| 120 | 1.35 | 2.05 |
| 240 | 1.39 | 2.13 |

Those skilled in the art would recognize that polymorphic forms of the present invention may be further characterized by a variety of other solid state spectroscopic techniques including, but not limited to, Raman spectroscopy, vibrational spectroscopy, polarized light microscopy (PLM), and solid state NMR, the ¹³C NMR and ¹H NMR (in a suitable solvent, e.g., in D₂O or DMSO-i¾) to evaluate the chemical structure, Dynamic Gravimetric Vapor Sorption (DVS) to evaluate the hygroscopicity, hot-stage optical microscopy to examine thermal transitions and/or chromatography (e.g., HPLC) in a suitable solvent to evaluate the purity. Products as described herein can be further analysed via Karl Fischer Titration (KF) to determine the water content.

According to another embodiment of the present invention, there is disclosed a process for preparing crystalline Form C1 of Ensifentrine, the process comprising:
a) Dissolving Ensifentrine base in a mixture of acid and water;
b) Treating with a suitable base;
c) Isolating the solid; and
d) Drying to obtain crystalline Form C1 of Ensifentrine.

In an embodiment, Ensifentrine base used in the processes of the present invention may be in any polymorphic form or in a mixture of any polymorphic forms such as hydrated, solvated, non-solvated or mixture of hydrated, solvated or non-solvated forms or amorphous thereof.

The starting material may be obtained by any method known in the art, such as the one described in U.S. Pat. No. 6,794,391 which is incorporated herein by reference or using the novel process of the present invention.

In an embodiment, acid used is selected from organic acid and inorganic acid.

In an embodiment, inorganic acid is selected from but not limited to hydrochloric acid, hydrobromic acid, hydroiodic acid, sulfuric acid, nitric acid, phosphoric acid and the like.

In an embodiment, organic acid is selected from but not limited to organic carboxylic acid salts such as acetic acid, lactic acid, citric acid, formic acid, oxalic acid, glutaric acid, malic acid, tartaric acid, fumaric acid, mandelic acid, uric acid, malic acid, maleic acid, benzoic acid and phthalic acid; and organic sulfonic acid such as methane sulfonic acid ethane sulfonic acid, benzene sulfonic acid, p-toluene sulfonic acid and camphor sulfonic acid and the like.

Preferably, acid used is hydrochloric acid, more preferably concentrated hydrochloric acid.

Preferably, Ensifentrine is stirred in a mixture of a concentrated hydrochloric acid and water.

Preferably, mixing is done at a temperature of 10 to 30° C., more preferably at 20 to 25° C.

Preferably, after mixing, heating is done at a suitable temperature and for a sufficient period to effect complete dissolution, preferably at about 40° C. to about 100° C., more preferably at 50° C. to about 80° C.

Optionally, the solution is filtered to remove the insoluble.

In an embodiment, base used is selected from organic and/or inorganic base.

The inorganic base may be selected from the group comprising of alkali or alkaline earth metal carbonates, such as cesium carbonate, sodium carbonate, potassium carbonate, magnesium carbonate, calcium carbonate, lithium carbonate or barium carbonate; alkali or alkaline earth metal hydroxides such as sodium hydroxide, potassium hydroxide, lithium hydroxide, magnesium hydroxide, calcium hydroxide, strontium hydroxide or barium hydroxide; alkali metal amides such sodium as amide, lithium diisopropylamide, lithium hexamethyldisilazide; alkali metal alkoxides such as sodium methoxide, sodium ethoxide, potassium t-butoxide; alkyllithiums such as t-BuLi; alkali metal hydrides such as sodium hydride, potassium hydride and 1,8-diazabicyclo[5.4.0]undec-7-ene.

Organic bases may be aliphatic or aromatic and may be selected from, but not limited to ethyl amine, diethyl amine, triethyl amine, triethanolamine, diisopropyl amine, N,N-diisopropylethylamine, pyridine, picoline, piperidine, dicyclohexylamine or cyclohexyl amine.

Most preferably, the base is selected from the group comprising of sodium hydroxide, and/or potassium hydroxide.

In an embodiment, Ensifentrine solution is treated with aqueous sodium hydroxide solution at a temperature, preferably at about 0° C. to about 40° C., more preferably at 10° C. to about 30° C., over a period of ranging from around 20 minutes to around 2 hours, preferably from around 30 minutes to around 1.5 hours, more preferably for a time of around 40 minutes to 1.2 hour, for example over a period of around 60 minutes.

Optionally, the aqueous sodium hydroxide solution, is first seeded with Ensifentrine to obtain slurry of crystalline Form C1 of Ensifentrine. Preferably, the seeding is done with crystalline Form C1 of Ensifentrine.

In an embodiment pH of the reaction mixture is adjusted preferably at about 8 to 13, more preferably at about 9 to 12.

Preferably, the process further comprises a stirring step. Preferably, the stirring is for about 15 minutes to about 3 hours, more preferably for about 30 minutes to about 2 hours. Preferably, the stirring is at about 0° C. to about 30° C., preferably at about 10° C. to about 30° C.

Preferably, the obtained solid form is isolated. Preferably, the isolation is done by centrifugation.

The drying may be done in a vacuum oven preferably at a temperature of about 30° C. to about 65° C., more preferably at 40° C. to about 60° C. Preferably, drying is about 5 hours to about 20 hours, more preferably for about 10 hours to about 15 hours.

The crystalline Form C1 of Ensifentrine, obtained as per the present invention is substantially free from other forms of Ensifentrine. “Substantially free” from other forms of Ensifentrine shall be understood to mean that crystalline Form C1 of Ensifentrine contains less than 10%, preferably less than 5%, of any other forms of Ensifentrine and less than 1% of other impurities.

According to another aspect of the present invention, there is provided a process for preparing crystalline Form C2 of Ensifentrine, the process comprising:
a) Chilling the suitable solvent at a lower temperature;
b) Adding Ensifentrine to this prechilled solvent;
c) Stirring the reaction mass;
d) Isolating the solid; and
e) Drying the solid to obtain crystalline Form C2 of Ensifentrine.

The suitable solvent used can be water, alcohol, ketones, ethers, nitriles, hydrocarbons, esters, aprotic polar solvents or mixture thereof preferably esters, ethers, and ketones.

The alcohol can be selected from but not limited to methanol, ethanol, n-propanol, isopropanol, n-butanol, isobutanol, tert-butanol and the like.

Ketones can be selected from but not limited to acetone, ethyl methyl ketone, methyl isobutyl ketone, methyl isopropyl ketone and the like.

Ethers can be selected from but not limited to tetrahydrofuran, 2-methyl tetrahydrofuran, t-butyl methyl ether, Di-isopropyl ether, Di ethyl ether, Petroleum ether and the like.

Nitriles can be selected from but not limited to acetonitrile, propionitrile, butyronitrile and the like.

Hydrocarbons including halogenated hydrocarbons can be selected from but not limited to toluene, xylene, dichloromethane, chlorobenzene and the like.

Aprotic polar solvents selected from but not limited to sulfolane, dimethyl sulfoxide, N-methyl pyrrolidone and the like.

Esters can be selected from but not limited to ethyl acetate, isopropyl acetate, Ethyl formate, amyl acetate, n-butyl acetate, iso butyl acetate, n-propyl acetate and the like.

The lower temperature may be selected from the range of −20 to 20° C., preferably −15 to 10° C. and more preferably in the range of −10 to 0° C.

Preferably, the process further comprises a stirring step. Preferably, the stirring is for about 15 minutes to about 3 hours, more preferably for about 30 minutes to about 2 hours.

Preferably, the obtained solid form is isolated. Preferably, the isolation is done by centrifugation.

The drying may be done in a vacuum tray drier preferably at a temperature of about 40° C. to about 80° C., more preferably at 50° C. to about 70° C. Preferably, drying is about 5 hours to about 20 hours, more preferably for about 10 hours to about 15 hours.

The crystalline Form C2 of Ensifentrine, obtained as per the present invention is substantially free from other forms of Ensifentrine. “Substantially free” from other forms of Ensifentrine shall be understood to mean that the crystalline Form C2 of Ensifentrine contains less than 10%, preferably less than 5%, of any other forms of Ensifentrine and less than 1% of other impurities.

According to another aspect of the present invention, there is provided a process for preparing crystalline Form C3 of Ensifentrine, the process comprising:
a) Dissolving Ensifentrine in a mixture of acid and suitable solvent;
b) Cooling the solution to −5 to 10° C.;
c) Treating the cooled solution with freshly prepared mixture of suitable base in a suitable solvent;
d) Isolating the solid;
e) Treating the isolated solid with pre-chilled water;
f) Isolating the solid; and
g) Drying the solid at suitable temperature for suitable period of time to obtain crystalline Form C3 of Ensifentrine.

In an embodiment, suitable solvents used in step a) include but not limited to ester, alcohol, ether, nitrile, hydrocarbons. Esters such as methyl acetate, ethyl acetate, butyl acetate, isopropyl acetate, isobutyl acetate or mixtures thereof; alcohols can be C1 to C5 alcoholic solvents such as methanol, ethanol, isopropanol, n-propanol, isobutanol, n-butanol, tert-butanol, 1,2-dimethoxy ethanol, 2-methoxy ethanol, 2-ethoxy ethanol and mixtures thereof; ethers such as diethyl ether, isopropyl ether, 1,4-dioxane, petroleum ether, methyl tertiary butyl ether (MTBE), and cyclic ethers such as THF, 2-methyl THF; nitriles such as acetonitrile, ethane nitrile, butyronitrile and mixtures thereof and hydrocarbons such as cyclohexane, hexane, n-heptane, chlorobenzene, dichloroethane, dichloromethane and mixtures thereof.

Acids used can be inorganic acid or organic acid.

In an embodiment, inorganic acid is selected from but not limited to hydrochloric acid, hydrobromic acid, hydroiodic acid. sulfuric acid, nitric acid, phosphoric acid and the like.

In an embodiment, organic acid is selected from but not limited to formic acid, acetic acid, lactic acid, citric acid, oxalic acid, glutaric acid, malic acid, tartaric acid, fumaric acid, mandelic acid, uric acid, malic acid, maleic acid, benzoic acid and phthalic acid; and organic sulfonic acids such as methane sulfonic acid ethane sulfonic acid, benzene sulfonic acid, p-toluene sulfonic acid and camphor sulfonic acid and the like

Preferably, the acid used is formic acid and the solvent is ethyl acetate.

Preferably, Ensifentrine is stirred in a mixture of a formic acid and ethyl acetate.

Preferably, mixing is done at ambient temperature, more preferably at a temperature of 20 to 30° C.

Optionally, the solution is filtered to remove the insoluble.

In an embodiment, the base used in step c) is selected from organic and inorganic base.

The inorganic base may be selected from the group comprising of ammonia, such as gaseous ammonia, aqueous ammonia or alcoholic ammonia, alkali or alkaline earth metal carbonates, such as cesium carbonate, sodium carbonate, potassium carbonate, magnesium carbonate, calcium carbonate, lithium carbonate or barium carbonate; alkali or alkaline earth metal hydroxides such as sodium hydroxide, potassium hydroxide, lithium hydroxide, magnesium hydroxide, calcium hydroxide, strontium hydroxide or barium hydroxide; alkali metal amides such as sodium amide, lithium diisopropylamide, lithium hexamethyldisilazide; alkali metal alkoxides such as sodium methoxide, sodium ethoxide, potassium t-butoxide; alkyllithiums such as t-BuLi; alkali metal hydrides such as sodium hydride, potassium hydride and 1,8-diazabicyclo[5.4.0]undec-7-ene.

Organic bases may be aliphatic or aromatic and may be selected from, but not limited to ethyl amine, diethyl amine, triethyl amine, triethanolamine, diisopropyl amine, N,N-diisopropylethylamine, pyridine, picoline, piperidine, dicyclohexylamine or cyclohexyl amine.

Most preferably, the base used in the preparation of Ensifentrine polymorphic form C3 is methanolic ammonia.

Suitable solvent used in step c) can be but not limited to esters such as methyl acetate, ethyl acetate, butyl acetate, isopropyl acetate, ethyl formate, amyl acetate, n-butyl acetate, iso butyl acetate, or mixtures thereof; alcohols such as methanol, ethanol, isopropanol, n-propanol, isobutanol, n-butanol, tert-butanol, 1,2-dimethoxy ethanol, 2-methoxy ethanol, 2-ethoxy ethanol and mixtures thereof; ethers such as tetrahydrofuran, diethyl ether, isopropyl ether, petroleum ether, diisopropyl ether (DIPE), 1,4-dioxane, methyl tertiary butyl ether (MTBE), 2-methyl THF; nitriles such as acetonitrile, ethane nitrile, butyronitrile and mixtures thereof and hydrocarbon solvents selected from but not limited to C6 to C10 alkane solvents such as cyclohexane, hexane, n-heptane, halogenated hydrocarbons such as dichloromethane, dichloromethane, chlorobenzene and mixtures thereof.

Solid may be isolated by the methods known in the art such as filtration, centrifugation.

Suitable temperature of drying is about 40 to 55° C. and suitable time is about 8-12 hours.

In an embodiment, the invention provides process for preparation of Ensifentrine Polymorphic Form C3. According to this embodiment, Ensifentrine is dissolved in formic acid and Ethyl acetate preferably at temperature of 10 to 30° C., more preferably at a temperature of 20 to 30° C.

Preferably, the dissolution of Ensifentrine conducted in a mixture of formic acid and Ethyl acetate comprises a step of stirring. Preferably, the stirring is for about 15 minutes to about 3 hours, more preferably for about 30 minutes to about 2 hours. Preferably, the stirring is at about 0° C. to about 30° C., preferably at about 10° C. to about 30° C., to get clear solution.

In an embodiment, the solution is optionally filtered to make it particle free.

The solution thus obtained is preferably cooled to a temperature of 0-5° C. and maintained at the same temperature for about 15 to 25 minutes. Thereafter, freshly prepared mixture of Methanolic ammonia and Ethyl acetate is charged into the above pre-chilled solution and maintained the reaction mass preferably for up to 60 to 120 minutes at −5 to 10° C., to obtain solids.

The resultant solid thus obtained is preferably charged into the pre-chilled water at a temperature of about −5 to 10° C. and maintained the reaction mass for up to 45 to 90 minutes at the same temperature. The resultant solids thus obtained are isolated by filtration and may be dried in an air tray dryer preferably at a temperature of 40 to 55° C. for 1.5 to 2.5 hours and at a temperature of 40 to 60° C. for 7 to 9 hours to obtain Ensifentrine polymorphic Form C3.

The crystalline Form C3 of Ensifentrine, obtained according the present invention is substantially free from other forms of Ensifentrine. “Substantially free” from other forms of Ensifentrine shall be understood to mean that crystalline Form C3 of Ensifentrine contains less than 10%, preferably less than 5%, of any other forms of Ensifentrine and less than 1% of impurities.

The processes of invention may be used as a method for purifying any form of Ensifentrine, as well as for the preparation of the new polymorphic forms.

According to another aspect of the present invention, there is provided a pharmaceutical composition comprising crystalline Forms C1, C2 and/or C3 of Ensifentrine as described above, together with one or more pharmaceutically acceptable excipients.

According to yet another aspect of the present invention there is provided use of crystalline Form C1, C2 and/or C3 of Ensifentrine as described above, in the preparation of a medicament useful in treating or preventing respiratory disorders such as cystic fibrosis, asthma and chronic obstructive pulmonary disease (COPD) in a mammal, wherein the mammal is human.

The starting material may be obtained by any method known in the art, such as the one described in U.S. Pat. No. 6,794,391 which is incorporated herein by reference or using the novel and improved process of the present invention.

Accordingly, the improved process for synthesizing Ensifentrine base comprises the steps of;
a) Condensing 2-Chloro-9,10-dimethoxy-6,7-dihydro-4H-pyrimido[6,1-a]isoquinolin-4-one with 2,4,6-trimethylaniline in a suitable solvent at a temperature in the range of 75 to 90° C. to obtain (E)-2-(mesitylimino)-9,10-dimethoxy2,3,6,7-tetrahydro-4H-pyrimido[6,1-a]isoquinolin-4-one;
b) Condensing (E)-2-(mesitylimino)-9,10-dimethoxy2,3,6,7-tetrahydro-4H-pyrimido[6,1-a]isoquinolin-4-one of step a) with N-(2-bromoethyl)phthalimide in a suitable solvent and in presence of catalyst and a base at a temperature in the range of 105 to 125° C. to (E)-2-(2-(2-(mesitylimino)-9,10-dimethoxy-4-oxo-6,7-dihydro-2H-pyrimido[6,1-a] isoquinolin-3(4H)-yl) ethyl) isoindoline-1,3-dione;
c) Purifying the material by dissolving the material obtained of step b) in water and alcohol mixture followed by addition of the base, stirring at a temperature in the range of 20 to 35° C. to obtain purified (E)-2-(2-(2-(mesitylimino)-9,10-dimethoxy-4-oxo-6,7-dihydro-2H-pyrimido[6,1-a] isoquinolin-3(4H)-yl) ethyl) isoindoline-1,3-dione;

d) Reacting purified (E)-2-(2-(2-(mesitylimino)-9,10-dimethoxy-4-oxo-6,7-dihydro-2H-pyrimido [6,1-a] isoquinolin-3(4H)-yl) ethyl) isoindoline-1,3-dione of step c) with 40% methyl amine in suitable solvent at a temperature in the range of 35 to 50° C. to obtain (E)-3-(2-aminoethyl)-2-(mesitylimino)-9, 10-dimethoxy-2,3,6,7-tetrahydro-4H-pyrimido[6,1-a] isoquinolin-4-one; and
e) Reacting (E)-3-(2-aminoethyl)-2-(mesitylimino)-9,10-dimethoxy-2,3,6,7-tetrahydro-4H-pyrimido[6,1-a]isoquinolin-4-one of step d) with sodium cyanate in suitable solvent and acid at a temperature in the range of 60 to 75° C. to obtain Ensifentrine.

The suitable solvent used in step a) or b) or d) or e) may include but not limited to an acid, ester, alcohol, ether, nitrile, hydrocarbons. Acids used can be inorganic acid or organic acid. Inorganic acid is selected from but not limited to hydrochloric acid, hydrobromic acid, hydroiodic acid, sulfuric acid, nitric acid, phosphoric acid and the like.

Organic acid is selected from but not limited to formic acid, acetic acid, lactic acid, citric acid, oxalic acid, glutaric acid, malic acid, tartaric acid, fumaric acid, mandelic acid, uric acid, malic acid, maleic acid, benzoic acid and phthalic acid; and organic sulfonic acids such as methane sulfonic acid ethane sulfonic acid, benzene sulfonic acid, p-toluene sulfonic acid and camphor sulfonic acid and the like.

Esters such as methyl acetate, ethyl acetate, butyl acetate, isopropyl acetate, isobutyl acetate or mixtures thereof; alcohols can be C1 to C5 alcoholic solvents such as methanol, ethanol, isopropanol, n-propanol, isobutanol, n-butanol, tert-butanol, 1,2-dimethoxy ethanol, 2-methoxy ethanol, 2-ethoxy ethanol and mixtures thereof; ethers such as diethyl ether, isopropyl ether, 1,4-dioxane, petroleum ether, methyl tertiary butyl ether (MTBE), and cyclic ethers such as THF, 2-methyl THF; nitriles such as acetonitrile, ethane nitrile, butyronitrile and mixtures thereof and hydrocarbons such as cyclohexane, hexane, n-heptane, chlorobenzene, dichloroethane, dichloromethane and mixtures thereof.

The suitable catalyst used in step b) may include but not limited to sodium iodide, potassium iodide, tetrabutyl ammonium iodide and the like.

The suitable base used in step b) may include but not limited to potassium carbonate, triethyl amine, potassium t-butoxide, lithium carbonate, sodium hexamethyl disilazane, 1,8-Diazabicyclo[5.4.0]undec-7-ene, sodium hydroxide, sodium hydride, potassium hydroxide.

The suitable alcohol used in step c) may include but not limited to methanol, ethanol, isopropanol. N-propanol, n-butanol, iobutanol, tert-butanol and the like.

The suitable acid used in step e) may be an inorganic acid is selected from but not limited to hydrochloric acid, hydrobromic acid, hydroiodic acid, sulfuric acid, nitric acid, phosphoric acid and the like.

The schematic representation of the process is as follows:

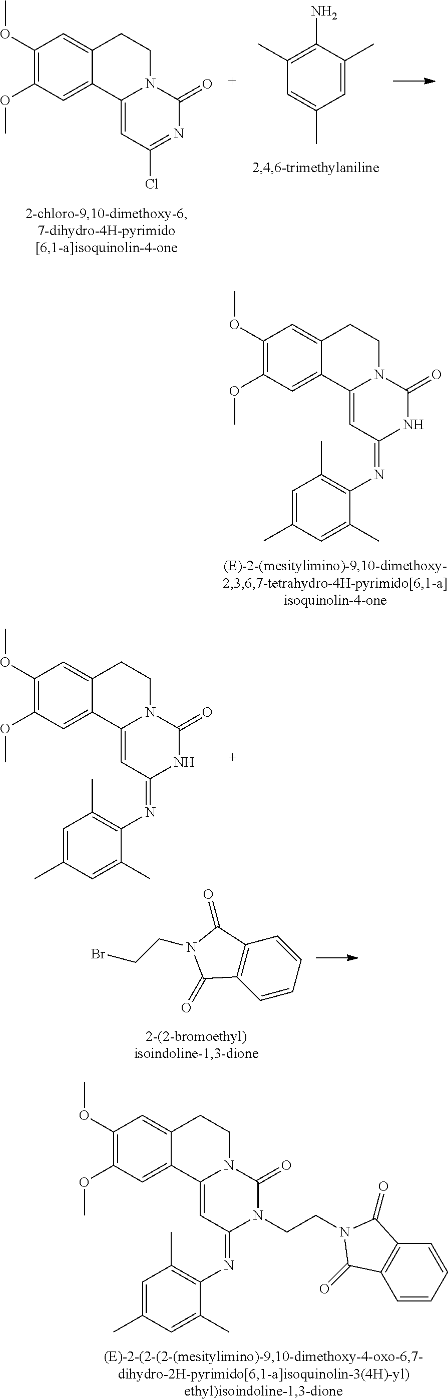

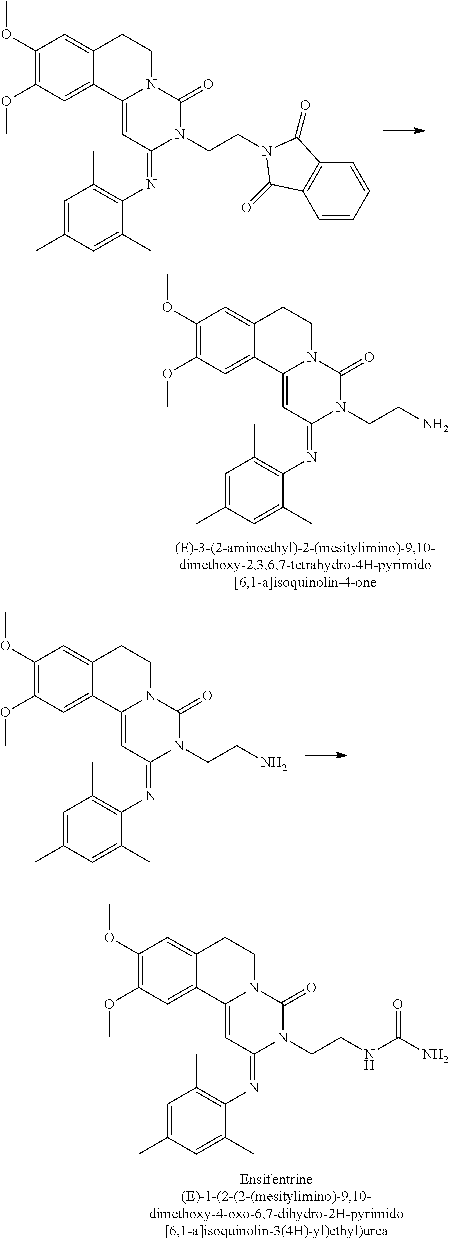

The inventors observed that the step of obtaining (E)-2-(2-(2-(mesitylimino)-9,10-dimethoxy-4-oxo-6,7-dihydro-2H-pyrimido[6,1-a] isoquinolin-3(4H)-yl) ethyl) isoindoline-1,3-dione is very lengthy and requires around 96 hours of reflux which is not feasible and safe at large scale manufacture of the product at plant level and also gives lesser yield. The inventors tried various combinations of solvents as well as various amounts of reagents. Various combinations of catalyst such as sodium iodide, potassium iodide, tetrabutyl ammonium iodide, base such as potassium carbonate, triethyl amine, potassium t-butoxide, lithium carbonate, sodium hexamethyl disilazane, 1,8-Diazabicyclo[5.4.0]undec-7-ene, sodium hydroxide, sodium hydride, potassium hydroxide and solvents such as toluene, acetone, dimethyl formamide, acetonitrile, sulfolane, ethyl methyl ketone and appropriate amounts were experimented. During the course of these experiments, inventors found that the combination of the catalyst, solvents and base mentioned in Table-1 below provide good yields in lesser time period compared to prior art.

TABLE 1

| | | | | Solvent | % Purity | |
|---|---|---|---|---|---|---|
| | A: Eq of | B: Eq of | C: Eq of | Combination | (ENF- | |
| Sr. | bromo | Sodium | Potassium | Toluene + | Phthalimide | Yield |
| No. | phthalimide | iodide | Carbonate | Chlorobenzene | compound) | (Efficiency %) |
| 1 | 3 | 2 | 5.6 | 5 + 5 | 97.2 | 69.44% |
| 2 | 3 | 2 | 5.66 | 5 + 5 | 98.86 | 72.03% |
| 3 | 2 | 1 | 4.5 | 5 + 5 | 91.25 | 36.39% |
| 4 | 4 | 1 | 4.5 | 5 + 5 | 89.983 | 56.15% |
| 5 | 4 | 1 | 6.5 | 5 + 5 | 90.846 | 57.82% |
| 6 | 2 | 3 | 6.5 | 5 + 5 | 94.878 | 46.59% |
| 7 | 4 | 3 | 6.5 | 5 + 5 | 89.346 | 65.72% |

All the experiments were carried out at 110 to 120° C.

From the experimental results mentioned in Sr. 1 & 2 above, it was observed that with the use of solvents toluene and chlorobenzene in 1:1 ratio; the yield and reaction time both were improved. The reaction time was drastically reduced to 25 hours from 96 hours. Further, the yield obtained earlier has 13.5% efficiency whereas the efficiency with the process of the present invention is 60-70%.

In an embodiment, Ensifentrine obtained by the improved process of the present invention may be further converted to its suitable solid crystalline “C1”, “C2” or “C3” forms by the process described above.

The following examples are provided for purposes of illustration, not limitation.

### EXAMPLES

### Example 1

### Preparation and Characterization of Crystalline Form C1 of Ensifentrine

10 g of Ensifentrine Form I, was dissolved in a mixture of DI water (300 ml) and concentrated Hydrochloric acid (10 ml) at 50-55° C. The solution was filtered through hyflo bed. The resultant clear filtrate was basified to pH 10-11, with aqueous sodium hydroxide solution (8 gm sodium hydroxide was dissolved in 10 ml DI water) at 25-30° C. The reaction mass was stirred further for 30 minutes. The solids were isolated by filtration and dried at 50-55° C. under vacuum for 10-12 hours to yield 6.5-7 gm of the title compound.

The solid was analyzed by XRPD, and identified as crystalline Form C1 of Ensifentrine as depicted in FIG. 1.

### Example 2

### Preparation and Characterization of Crystalline Form C2 of Ensifentrine

16 ml (4 volumes) of Isopropyl acetate was charged into the round bottom flask and the solvent was cooled to −10° C. 4 gm of Ensifentrine amorphous material was charged into the pre-chilled Isopropyl acetate and the reaction mass was stirred for 1 hour at −10° C. The resulting solid was isolated by filtration and dried in Vacuum Tray Drier at 40° C. for 3 hours and 60° C. for 11 hours to obtain 3.2 g of the title compound.

The solid was analyzed by XRPD and identified as crystalline Form C2 of Ensifentrine as depicted in FIG. 6.

### Example 3

### Preparation and Characterization of Crystalline Form C2 of Ensifentrine

4 ml (4 volumes) of Tetrahydrofuran was charged into the round bottom flask and the solvent was cooled to −10° C. 1 gm of Ensifentrine amorphous material was charged into the pre-chilled Tetrahydrofuran and the reaction mass was stirred for 1 hour at −10° C. The resulting solid was isolated by filtration and dried in Vacuum Tray Drier at 60° C. for 8 hours to obtain 0.7 g of the title compound.

The solid was analyzed by XRPD and identified as crystalline Form C2 of Ensifentrine as depicted in FIG. 7.

### Example 4

### Preparation and Characterization of Crystalline Form C3 of Ensifentrine

Charged 2 g of Ensifentrine, 2 mL of formic acid and 15 mL of Ethyl acetate into a clean round bottom flask (RBF) and stirred for 10 mins at room temperature to get clear solution. Filtered the above solution to make it particle free and transferred it into another RBF. Cooled the solution to 0-5° C. and maintained at 0-5° C. for 20 mins. Slowly charged the freshly prepared mixture of 10 mL of Methanolic ammonia and 15 mL of Ethyl acetate into the above pre-chilled solution and maintained the reaction mass for 90 min at 0-5° C. The resultant solids were isolated by filtration and suck dried the material for 30 mins. The resultant wet material was charged into the pre-chilled water (charged 19 ml of water into a clean RBF and cooled the water RBF at 0-5° C. for 20 min) and maintained the reaction mass for 1 hr at 0-5° C. The resultant solids were isolated by filtration and suck dried the material. Dried the material in ATD at 45° C. for 2 hrs and 50° C. for 8 hrs to obtain the title compound.
Yield: Dry weight: 0.8 g.

The solid was analyzed by XRPD and identified as crystalline Form C3 of Ensifentrine as depicted in FIG. 10.

### Example 5

### Preparation of Free Ensifentrine Base, (N-{2-[(2E)-2-(mesitylimino)-9,10-dimethoxy-4-oxo-6,7-dihydro-2H-pyrimido[6,1-a]-isoquinolin-3 (4H)-yl]ethyl}urea).

100 g of 2-chloro-9,10-dimethoxy-6,7-dihydro-4H-pyrimido[6,1-a]isoquinolin-4-one compound was taken in a clean and dry reactor and 7 litre of isopropyl alcohol (IPA) was charged into it. The reaction mass was stirred for 5 minutes followed by addition of 136.88 g of 2,4,6-trimethyl aniline, and 1 litre of IPA. The reaction mass was heated to 80-85° C. and stirred for 24 hours. After completion of the reaction, reaction mass was cooled to 25-30° C., stirred and filtered. The filtered material was washed with IPA and dried to get (E)-2-(mesitylimino)-9,10-dimethoxy2,3,6,7-tetrahydro-4H-pyrimido[6,1-a]isoquinolin-4-one.

100 g of the material obtained in above step was charged into a clean and dry reactor followed by addition of 500 ml toluene and stirred for 5 minutes. 200 g of potassium carbonate was charged into the reaction mass followed by addition of 200 g of N-(2-bromoethyl) phthalimide. Further, 80 g of sodium Iodide and 500 ml of chlorobenzene were charged to the reaction mass. The reaction mass was then heated to reflux at 110-120° C. and stirred for about 20 hours. After the completion of the reaction, the mass was cooled to 25-30° C. 10 g of Hyflo was charged to it and stirred for 1 hour. The reaction mass was filtered, washed with 300 ml of toluene and suck dried for 15 minutes and washed with 300 ml of toluene and suck dried. Toluene layer was distilled out under vacuum below 60° C. 150 ml of Methanol added to the reaction mass, heated and then distilled under vacuum. The process of addition of methanol repeated.

400 ml of methanol was further added into it and stirred for 15 minutes. The reaction mass was cooled to 25-30° C., stirred for 1 hour, filtered and washed with 100 ml chilled methanol and then suck dried for 2 hours to get crude (E)-2-(2-(2-(mesitylimino)-9,10-dimethoxy-4-oxo-6,7-dihydro-2H-pyrimido[6,1-a]isoquinolin-3(4H)-yl) ethyl) isoindoline-1,3-dione.

Purification—220 g of the above material was charged into the clean and dry flask followed by 1100 ml water and 1100 ml methanol. A solution of 44 g sodium hydroxide was prepared with 220 ml water and cooled to 25-30° C. and this NaOH solution was added to the reaction mass. The reaction mass was then stirred for 2 hours at ambient temperature (25-30° C.), filtered, washed with 440 ml water. Dried under vacuum oven at 50-55° C. for 10 hours.
Yield: 100 g, Efficiency: about 70%
Purity: 99%

100 g of (E)-2-(2-(2-(mesitylimino)-9,10-dimethoxy-4-oxo-6,7-dihydro-2H-pyrimido[6,1-a] isoquinolin-3(4H)-yl)ethyl)isoindoline-1,3-dione was charged into the clean flask followed by addition of 700 ml of methanol and stirred for 5 minutes. 200 ml of 40% aqueous methyl amine solution was charged into the reaction mass and stirred for 10 minutes followed by heating to 40-45° C. and stirring reaction mass for 4 hours.

After completion of the reaction, the reaction mass was distilled out under vacuum to get an oil to which 1000 ml water was added and extracted with 3×300 ml of methylene dichloride (MDC). Solvent was distilled out completely to get (E)-3-(2-aminoethyl)-2-(mesitylimino)-9,10-dimethoxy-2,3,6,7-tetrahydro-4H-pyrimido[6,1-a]isoquinolin-4-one as a yellow foamy material.

50 g of the above material was charged into clean and dry flask followed addition of 1500 ml of water and stirred for 5 minutes at 20-30° C. To this, 230 ml 1N HCl was charged. 15 g Sodium cyanate was dissolved into 500 ml water and this solution was added slowly into the reaction mass over one hour. It was then heated to 65-70° C. and stirred for about 5-6 hours. After completion of the reaction, the reaction mass was cooled to 25-30° C. and basified to pH 8-9 using 2N NaOH and stirred for 1 hour. The reaction mass was then filtered through Buchner funnel, washed with 100 ml water and suck dried. Further dried in vacuum oven at 60° C. for 8-10 hours to get Ensifentrine. The so obtained crude material is purified by the process known in the art.
Yield: 40 g
Purity: 99%

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

We claim:

1. A solid crystalline polymorphic Form C3 of Ensifentrine characterized by powder X-ray diffraction pattern having at least one peak selected from those at 5.6, 6.6, 9.7, 13.5 and 16.3±0.2° 2θ.

2. The solid crystalline polymorphic Form C3 of Ensifentrine as claimed in claim 1 characterized by at least one of:
powder X-ray diffraction pattern as depicted in FIG. 10;
a DSC, substantially as depicted in FIG. 11;
a TGA, substantially as depicted in FIG. 12; or
Infrared spectrometry, substantially as depicted in FIG. 13.

3-5. (canceled)

6. The solid crystalline polymorphic Form C3 of Ensifentrine as claimed in claim 1, as hydrated or solvated or anhydrous form.

7. A process for preparing crystalline polymorphic Form C3 of Ensifentrine, the process comprising;
a) Dissolving Ensifentrine base in a mixture of acid and suitable solvent;
b) Cooling the solution to −5 to 10° C.;
c) Treating the cooled solution with freshly prepared mixture of suitable base in a suitable solvent;
d) Isolating the solid;
e) Treating the isolated solid with pre-chilled water;
f) Isolating the solid; and
g) Drying the solid at suitable temperature for suitable period of time to obtain crystalline Form C3 of Ensifentrine.

8. The process as claimed in claim 7, wherein the acid in step a) is selected from:
organic acids, which is selected from the group consisting of: formic acid, acetic acid, lactic acid, citric acid, oxalic acid, glutaric acid, malic acid, tartaric acid, fumaric acid, mandelic acid, uric acid, malic acid, maleic acid, benzoic acid, or phthalic acid;
organic sulfonic acids selected from: methane sulfonic acid, ethane sulfonic acid, benzene sulfonic acid, p-toluene sulfonic acid, camphor sulfonic acid, or mixtures thereof; or
inorganic acid selected from; hydrochloric acid, hydrobromic acid, hydroiodic acid, sulfuric acid, nitric acid, phosphoric acid, or combinations thereof.

9. The process as claimed in claim 7, wherein the solvent in step a) is selected from: ester, alcohol, ether, nitrile, hydrocarbons alone, or combinations thereof.

10. The process as claimed in claim 9, wherein the solvent is selected from:
esters which is selected from: methyl acetate, ethyl acetate, butyl acetate, isopropyl acetate, isobutyl acetate, or mixtures thereof;
alcohols which is selected from: methanol, ethanol, isopropanol, n-propanol, isobutanol, n-butanol, tert-butanol, 1,2-dimethoxy ethanol, 2-methoxy ethanol, 2-ethoxy ethanol, or mixtures thereof;
ethers which is selected from: diethyl ether, isopropyl ether, 1,4-dioxane, petroleum ether, or methyl tertiary butyl ether (MTBE), or cyclic ethers which is selected from: THF, 2-methyl THF;
nitriles, acetonitrile, ethane nitrile, butyronitrile, or mixtures thereof, or hydrocarbons which is selected from: cyclohexane, hexane, n-heptane, chlorobenzene, dichloroethane, dichloromethane, or mixtures thereof.

11. The process as claimed in claim 7, wherein the acid is formic acid and the solvent is ethyl acetate.

12. The process as claimed in claim 7, wherein the base in step c) is selected from:
inorganic base, which is selected from the group consisting of: ammonia gaseous ammonia, aqueous ammonia or alcoholic ammonia, alkali or alkaline earth metal carbonates, cesium carbonate, sodium carbonate, potassium carbonate, magnesium carbonate, calcium carbonate, lithium carbonate, or barium carbonate;
alkali or alkaline earth metal hydroxides, sodium hydroxide, potassium hydroxide, lithium hydroxide, magnesium hydroxide, calcium hydroxide, strontium hydroxide, or barium hydroxide;
alkali metal amides selected from: sodium amide, lithium diisopropylamide, or lithium hexamethyldisilazide;
alkali metal alkoxides selected from: sodium methoxide, sodium ethoxide, or potassium t-butoxide;
alkyllithiums which comprises—BuLi;
alkali metal hydrides selected from: sodium hydride, potassium hydride, or 1,8-diazabicyclo[5.4.0]undec-7-ene;
organic base selected from aliphatic or aromatic base which is selected from: ethyl amine, diethyl amine, triethyl amine, triethanolamine, diisopropyl amine, N,N-diisopropylethylamine, pyridine, picoline, piperidine, dicyclohexylamine, or cyclohexyl amine; or
a mixture thereof, preferably the base is methanolic ammonia.

13. (canceled)

14. The process as claimed in claim 7, wherein the solvent in step c) is selected from the group consisting of:
esters which comprises: methyl acetate, ethyl acetate, butyl acetate, isopropyl acetate, ethyl formate, amyl acetate, n-butyl acetate, iso butyl acetate, or mixtures thereof;
alcohols which comprises: methanol, ethanol, isopropanol, n-propanol, isobutanol, n-butanol, tert-butanol, 1,2-dimethoxy ethanol, 2-methoxy ethanol, 2-ethoxy ethanol, or mixtures thereof;
ethers which comprises: tetrahydrofuran, diethyl ether, isopropyl ether, petroleum ether, diisopropyl ether (DIPE), 1,4-dioxane, methyl tertiary butyl ether (MTBE), 2-methyl THF;
nitrile solvents which comprises: acetonitrile, ethane nitrile, butyronitrile, or mixtures thereof, and
hydrocarbons selected from C6 to C10 alkane solvents which comprises: cyclohexane, hexane, n-heptane, halogenated hydrocarbons which comprises: dichloromethane, dichloroethane, chlorobenzene, or mixtures thereof.

15. The process as claimed in claim 7, further comprising at least one of the following features:
wherein the temperature for step a) ranges between 10° C. to 30° C.; or
wherein the temperature for drying in step g) is in the range of 40° C. to 60° C. and the time period is in the range of 7 to 9 hours.

16. (canceled)

17. The process for preparing crystalline Form C3 of Ensifentrine as claimed in claim 7, comprising:
a) Dissolving Ensifentrine base in a mixture of formic acid and Ethyl acetate at a temperature ranging between 10° C. to 30° C.;
b) Cooling at a temperature ranging between −5 to 10° C.;
c) Treating the cooled solution with freshly prepared mixture of Methanolic ammonia and Ethyl acetate;
d) Isolating the solid;
e) Treating the isolated solid with pre-chilled water;
f) Isolating the solid; and
g) Drying the solid at a temperature ranging between 40° C. to 55° C. for 1.5 to 2.5 hours and at a temperature ranging between 40 to 60° C. for 7 to 9 hours to obtain crystalline Form C3 of Ensifentrine.

18. The process as claimed in claim 7, wherein Ensifentrine contains less than 10%, preferably less than 5%, of any other forms of Ensifentrine, and less than 1% of impurities.

19. A pharmaceutical composition comprising the crystalline polymorphic Form C3 of Ensifentrine as claimed in claim 1, together with one or more pharmaceutically acceptable excipients.

20. A method to treat or prevent respiratory disorders selected from: cystic fibrosis, asthma, or chronic obstructive pulmonary disease (COPD) in a mammal, comprising: administering to said mammal an effective amount of a crystalline polymorphic Form C3 of Ensifentrine as claimed in claim 1.

21. (canceled)

22. A process for preparing Ensifentrine free base comprising;
a) Condensing 2-Chloro-9,10-dimethoxy-6,7-dihydro-4H-pyrimido[6,1-a] isoquinolin-4-one with 2,4,6-trimethylaniline in a suitable solvent at a temperature in the range of 75 to 90° C. to obtain (E)-2-(mesitylimino)-9,10-dimethoxy2,3,6,7-tetrahydro-4H-pyrimido[6,1-a]isoquinolin-4-one;
b) Condensing (E)-2-(mesitylimino)-9,10-dimethoxy2,3,6,7-tetrahydro-4H-pyrimido[6,1-a]isoquinolin-4-one of step a) with N-(2-bromoethyl) phthalimide in a suitable solvent in presence of catalyst and the base at a temperature in the range of 105 to 125° C. to obtain (E)-2-(2-(2-(mesitylimino)-9,10-dimethoxy-4-oxo-6,7-dihydro-2H-pyrimido[6,1-a] isoquinolin-3(4H)-yl) ethyl) isoindoline-1,3-dione;
c) Purifying the material by dissolving the material obtained in step b) in water and alcohol mixture followed by addition of base, stirring at a temperature in the range of 20 to 35° C. to obtain purified (E)-2-(2-(2-(mesitylimino)-9,10-dimethoxy-4-oxo-6,7-dihydro-2H-pyrimido[6,1-a]isoquinolin-3(4H)-yl) ethyl) isoindoline-1,3-dione;
d) Reacting purified (E)-2-(2-(2-(mesitylimino)-9,10-dimethoxy-4-oxo-6,7-dihydro-2H-pyrimido[6,1-a]isoquinolin-3(4H)-yl) ethyl) isoindoline-1,3-dione of step c) with 40% methyl amine in suitable solvent at a temperature in the range of 35 to 50° C. to get (E)-3-(2-aminoethyl)-2-(mesitylimino)-9,10-dimethoxy-2,3,6,7-tetrahydro-4H-pyrimido[6,1-a] isoquinolin-4-one-; and
e) Reacting (E)-3-(2-aminoethyl)-2-(mesitylimino)-9,10-dimethoxy-2,3,6,7-tetrahydro-4H-pyrimido[6,1-a]isoquinolin-4-one of step d) with sodium cyanate in suitable solvent and acid and at a temperature in the range of 60 to 75° C. to obtain Ensifentrine, optionally the Ensifentrine base obtained is further converted to solid crystalline polymorphic Form C3.

23. The process as claimed in claim 22, wherein the solvent for the process steps a), b), d) and e) is selected from: water, lower alcohols, which is selected from: methanol, ethanol, isopropanol, n-propanol, isobutanol, n-butanol, tert-butanol, 1,2-dimethoxy ethanol, 2-methoxy ethanol, 2-ethoxy ethanol, or mixtures thereof,; aromatic hydrocarbon selected from: toluene, xylene, or mixtures thereof; halogenated hydrocarbon selected from: dichloromethane, dichloroethane, chlorobenzene, or mixtures thereof; acids selected from: hydrochloric acid, hydrobromic acid, hydroiodic acid, sulfuric acid, nitric acid, phosphoric acid, formic acid, acetic acid, lactic acid, citric acid, oxalic acid, glutaric acid, malic acid, tartaric acid, fumaric acid, mandelic acid, uric acid, malic acid, maleic acid, benzoic acid, phthalic acid, or mixtures thereof; and organic sulfonic acids selected from: methane sulfonic acid ethane sulfonic acid, benzene sulfonic acid, p-toluene sulfonic acid, camphor sulfonic acid or mixtures thereof; esters selected from: methyl acetate, ethyl acetate, butyl acetate, isopropyl acetate, isobutyl acetate, or mixtures thereof; ethers selected from: diethyl ether, isopropyl ether, 1,4-dioxane, petroleum ether, methyl tertiary butyl ether (MTBE) or mixtures thereof, or cyclic ethers selected from: tetrahydrofuan (THF), 2-methyl THF; or nitriles selected from:
acetonitrile, ethane nitrile, butyronitrile, or mixtures thereof.

24. The process as claimed in claim 23, further comprising at least one of the following features:
wherein the solvent for step (b) is a mixture of toluene and chlorobenzene in 1:1 ratio;
wherein the catalyst for step (b) is selected from sodium iodide, potassium iodide, tetrabutyl ammonium iodide, or mixtures thereof; or
wherein the base for step (b) is selected from potassium carbonate, triethyl amine, potassium t-butoxide, lithium carbonate, sodium hexamethyl disilazane, 1,8-Diazabicyclo[5.4.0]undec-7-ene, sodium hydroxide, sodium hydride, potassium hydroxide, or mixtures thereof.

25-26. (canceled)

27. The process as claimed in claim 22, further comprising at least one of the following features:
wherein the solvent for step (c) selected from methanol, ethanol, isopropanol. N-propanol, n-butanol, iobutanol, tert-butanol or mixtures thereof; or
wherein the acid for step (e) is an inorganic acid selected from hydrochloric acid, hydrobromic acid, hydroiodic acid, sulfuric acid, nitric acid, phosphoric acid, or mixtures thereof.

28. (canceled)

29. (canceled)

30. A pharmaceutical composition comprising Ensifentrine prepared by the process of claim 22 together with one or more pharmaceutically acceptable excipients.
